(19) United States
(12) Patent Application Publication
(10) Pub. No.: US 2016/0235577 A1
(43) Pub. Date: Aug. 18, 2016

(54) NASAL DILATOR

(71) Applicant: Lei WANG, Beijing (CN)

(72) Inventor: Wang Lei, Beijing (CN)

(21) Appl. No.: 15/027,981

(22) PCT Filed: Oct. 10, 2013
(86) PCT No.: PCT/CN2013/085002

§ 371 Date: Apr. 7, 2016

(51) Int. Cl. A61F 5/08

(52) U.S. Cl. CPC A61F 5/08

(57) ABSTRACT

A nasal dilator including: two ring-shaped brackets and a connecting band. The two ends of the connecting band are respectively connected to a ring-shaped bracket in a symmetrical manner, the two ends of the connecting band are both provided with an insertion part. The connecting band is inserted into insertion holes on the ring-shaped bracket respectively via the insertion parts, and an included angle formed between a central axis of the connecting band and a central axis of the nasal dilator is 45 to 85 degrees. The ring-shaped brackets and the connecting band of the nasal dilator are of an insertion-connection design, so that the ring-shaped brackets located inside the nasal passage can be made from a soft material, and the connecting band can be made from a material with relative high hardness and relative strong elasticity.

### TECHNICAL FIELD OF THE INVENTION

The present invention relates to a dilating device, and particularly to a nasal dilator.

### BACKGROUND OF THE INVENTION

At present, quite a few people suffer from symptoms related to nasal passage deformity, for example, swelling caused by deviation of nasal septum and allergic reaction, and such deformity may cause difficulty in breathing and a snore. A lower portion of a nasal cavity, namely, a position slightly upward at a nostril inlet, is called a hole cavity. The hole cavity is in a shape of an inward taper and reaches a narrow neck-like region which is called a small hole region (inner orifice of a nasal duct). Nasal obstruction generally occurs at the small hole. For different patients, swelling caused by allergic reaction or deviation of nasal septum or similar problems might often cause a blockage of the small hole region. Generally, relaxation of a wall of the small hole (namely, outer wall tissue of the nasal passage) causes draw-in of the outer wall tissue during breathing, which will cause the nasal passage to be substantially obstructed when the air passes through the passage. Such draw-in of the outer wall tissue is like a “one-way valve”, which obstructs air flow during inspiration. The obstruction of the nasal passage is apt to cause to snore at night, or cause a patient to wake up constantly at night because sufficient oxygen cannot be inhaled, thereby affecting normal sleep of the patient.

The chronic and serious nasal passage obstructions stated above are generally treated by correcting the nasal passage deformity through surgical procedure. However, the surgical procedure is generally costly and difficult to radically cure the nasal passage deformity.

Another method for solving the nasal passage deformity in a relatively conveniently way is to use a nasal dilator to facilitate breathing. U.S. Pat. No. USD575397 S1 discloses a nasal dilator which mainly comprises two irregular annular stents and a U-shaped connecting device, and which is of a unitary design. After the nasal dilator is implanted in the nasal passage, the two irregular annular stents support an inner wall of the small hole in the nostril from inside to avoid the draw-in of the outer wall tissue during inspiration, i.e., reduce extra air flow resistance upon inspiration. The U-shaped connecting device is used to clamp the nasal septum to thereby stabilize a position of the nasal dilator in the nasal passage.

Since the nasal dilator is of a unitary design, the following contradictions occur. If a harder material is employed, the annular stents will stimulate soft nasal passage tissues, causing itch of the nasal cavity, runny nose and inflammation; if a softer material is employed, the annular stents cannot effectively support the inner wall of the nasal passage, the U-shaped connecting device cannot be effectively fixed onto the nasal septum either and is apt to disengage, but whether the nasal dilator can be fixed effectively is crucial for application of the nasal dilator to prevent snoring at night; secondly, an angle between a plane on which the annular stent lies and a plane on which the U-shaped connecting device lies is 90 degrees, which requires the U-shaped connecting device to be relatively longer to ensure the two annular stents deep into a position nearby the small hole of the nasal passage. A top end of the U-shaped connecting device will stimulate a sensitive area of the nasal passage, causing the user uncomfortable.

### SUMMARY OF THE INVENTION

An object of the present invention is to solve the above technical problems existing in the existing nasal dilator. The present invention provides a nasal dilator. Annular stents and a connecting band in the nasal dilator employ a plug-in design so that the annular stents located in the nasal passage may be made of a soft material to prevent a stimulation to the nasal cavity; whereas the connecting band may be made of a material with a larger hardness and stronger elasticity so that the nasal dilator can clamp a nasal septum more effectively and disengagement of the nasal dilator from the nose during in-use can be prevented; when the nasal dilator is fixed to the nasal septum, plug-in portions of the connecting band are not in contact with the nasal septum, while sides of the annular stents adjacent to a central axis of the nasal dilator clamp the nasal septum, such that the elastic force of the connecting band can evenly act upon a surface of the nasal septum through a larger contact area and a softer medium, thereby reducing the stimulation to the nasal septum; a posture of the nasal dilator may be designed in a way that in the event of a shorter connecting band, the soft annular stents of the nasal dilator may still get deep to be adjacent to the nasal cavity small hole to support the inner wall of the nasal cavity, reducing the stimulation exerted by the connecting band to the nasal passage and enabling the user to be free of sting.

To achieve the above object, the present invention provides a nasal dilator comprising two annular stents and a connecting band, both ends of the connecting band being respectively and symmetrically connected to one annular stent, wherein a plug-in portion may be provided at each of both ends of the connecting band, and the connecting band may be plugged in a plug-in hole on each of the annular stents via the plug-in portions; an central axis of the connecting band may be at an angle of 45 degrees to 85 degrees to a central axis of the nasal dilator;

the connecting band may be made of a harder nontoxic material and have a height in a range of 8 mm-20 mm;

the annular stent may be made of a softer nontoxic material and may be a circular ring provided with 1-3 reinforcing rib(s).

As a further improvement of the above described technical solution, the plug-in portion may be a dumbbell-shaped structure which is thin in the middle and thick at both ends, a hole wall of the plug-in hole on the annular stent may be nested around a thinner portion of the plug-in portion in the middle, and thicker portions of the plug-in portion at both ends are stuck to both sides of the hole wall.

As a further improvement of the above described technical solution, the plug-in hole of the annular stent may be located on a side adjacent to the central axis of the nasal dilator.

As a further improvement of the above described technical solution, after the plug-in portion is inserted into the plug-in hole of the annular stent, an edge of a side of the annular stent adjacent to the central axis of the nasal dilator may be spaced apart a distance of 0.5-3 mm from an edge of the plug-in portion.

As a further improvement of the above described technical solution, the connecting band may be in a strip structure which is substantially U-shaped or C-shaped so that an angle between planes on which the two annular stents lie is 100 degrees to 170 degrees.

As a further improvement of the above described technical solution, a cross section of the annular stent may be fit for different shapes of inner cavities of nasal vestibules; a top end of the annular stent may be narrower and oriented towards a nose tip when placed in the nasal passage, a bottom end of the annular stent may be wider and oriented towards a face when placed in the nasal passage, a side of the annular stent adjacent to the central axis of the nasal dilator may be a straight edge or an edge with a small radian and oriented towards the nasal septum when placed in the nasal passage, a side of the annular stent away from the central axis of the nasal dilator may be an arcuate edge and oriented towards a nose wing when placed in the nasal passage to support an inner wall of the nasal passage.

As a further improvement of the above described technical solution, the nontoxic material may be selected from a group consisting of plastic, resin, nylon, silicone or compositions fabricated on the basis of these materials.

As a further improvement of the above described technical solution, the reinforcing ribs may be disposed in parallel with the connecting band.

As a further improvement of the above described technical solution, the reinforcing rib may be wider at a joint with an inner wall of the annular stent and thinner in the middle.

Advantages of the nasal dilator according to the present invention lie in the following:

1. the annular stents and the connecting band in the nasal dilator may employ a plug-in design so that the annular stents located in the nasal passage may be made of a softer material to prevent a stimulation to the nasal cavity; the connecting band may be made of a material with a larger hardness and stronger elasticity so that the nasal dilator clamps a nasal septum more effectively and disengagement of the nasal dilator from the nose during in-use can be prevented;

2. with reinforcing ribs provided on the annular stents extending from the nasal septum to the nose wing, effective support exerted by the annular stents to the nasal passage is ensured, the middle portion of the reinforcing rib is thinner and may elastically deform to prevent injury to the nasal passage due to excessive support, the reinforcing ribs are wider at joints with the inner wall of the annular stents to ensure even pressure on the annular stents and avoid the stimulation to the inner wall of the nasal cavity due to excessive local pressure;

3. the annular stents and the connecting band may be replaced conveniently according to different shapes of inner cavities of nasal vestibules to conform to ergonomics design;

4. since the central axis of the connecting band is at an angle of 45 degrees to 85 degrees to the central axis of the nasal dilator, the annular stent is in an inclined posture in the nose so that the connecting band need not to get deep in the nasal passage, thereby reducing the stimulation to the nasal passage and enabling more comfortable wearing, and the bottom end of the annular stent can extend to be adjacent to the nasal cavity small hole deep in the nasal passage to function as a support;

5. after the plug-in portion of the connecting band is inserted into the plug-in hole of the annular stent, an edge of a side of the annular stent adjacent to the central axis of the nasal dilator is spaced apart a distance of 0.5-3 mm from the edge of the plug-in portion. When the nasal dilator is fixed onto the nasal septum, the plug-in portions of the connecting band are not in contact with the nasal septum, while sides of the annular stents adjacent to the central axis of the nasal dilator clamp the nasal septum, such that an elastic force of the connecting band can evenly act upon a surface of the nasal septum through a larger contact area and a softer medium, thereby reducing the stimulation to the nasal septum;

6. the angle between planes on which the two annular stents lie is 100 degrees to 170 degrees so that it is ensured that a supporting force of the annular stents acts upon the nose wings in a forward direction so as to achieve an effective dilation effect.

### BRIEF DESCRIPTION OF THE DRAWINGS

FIG. 1 is a front view of a nasal dilator according to one embodiment of the present invention.

FIG. 2 is a top view of a nasal dilator according to one embodiment of the present invention.

FIG. 3 is a side view of a nasal dilator according to one embodiment of the present invention.

FIG. 4 is an in-use state diagram of a nasal dilator according to one embodiment of the present invention.

The drawing label:

| | | | | | |
|---|---|---|---|---|---|
| 1. | annular stent | 2. | connecting band | 3. | plug-in hole |
| 4. | plug-in portion | 5. | reinforcing rib | | |

### DETAILED DESCRIPTION OF EMBODIMENT

The structure of the present invention will be described in more detail in combination with figures and specific embodiments.

A nasal dilator according to an embodiment of the present invention may comprise: two annular stents 1 and a connecting band 2, both ends of the connecting band 2 are respectively and symmetrically connected to one annular stent 1; wherein, a plug-in portion 4 may be provided at each of both ends of the connecting band 2, the connecting band 2 may be plugged in a plug-in hole 3 on each of the annular stents 1 via the plug-in portions 4; a central axis of the connecting band 2 may be at an angle of 45 degrees to 85 degrees to a central axis of the nasal dilator; the connecting band 2 may be made of a harder and more elastic nontoxic material and have a height in a range of 8 mm-20 mm; the annular stents 1 may be made of a softer nontoxic material and may be a circular ring provided with 1-3 reinforcing rib(s) 5.

According to the structure of the above-mentioned nasal dilator, FIG. 1 shows a nasal dilator according to one embodiment of the present invention. In the present embodiment, the annular stent 1 may be provided with two reinforcing ribs 5, and the height of the connecting band 2 may progressively increase along with an increase of the inner cavity of the nasal vestibule. In addition, a thickness of the annular stent 1 may be 1 mm-3 mm. The plug-in hole of the annular stent 1 may be located on one side adjacent to the central axis of the nasal dilator (as shown by line i-i in FIG. 1). A cross section of the annular stent 1 may be fit for different shapes of the inner cavities of the nasal vestibules; a top end of the annular stent 1 may be narrow and oriented towards a nasal tip when placed in the nasal passage, a bottom end of the annular stent 1 may be wide and oriented towards a face when placed in the nasal passage, one side of the annular stent 1 adjacent to the central axis of the nasal dilator may be a straight edge or an edge with a small radian and oriented towards the nasal septum when placed in the nasal passage, and a side of the annular stent 1 away from the central axis of the nasal dilator may be an arcuate edge and oriented towards a nose wing when placed in the nasal passage to support an inner wall of the nasal passage. The reinforcing ribs 5 may be disposed in parallel with the connecting band 2 or at an angle of 0 degrees to 30 degrees to the connecting band 2.

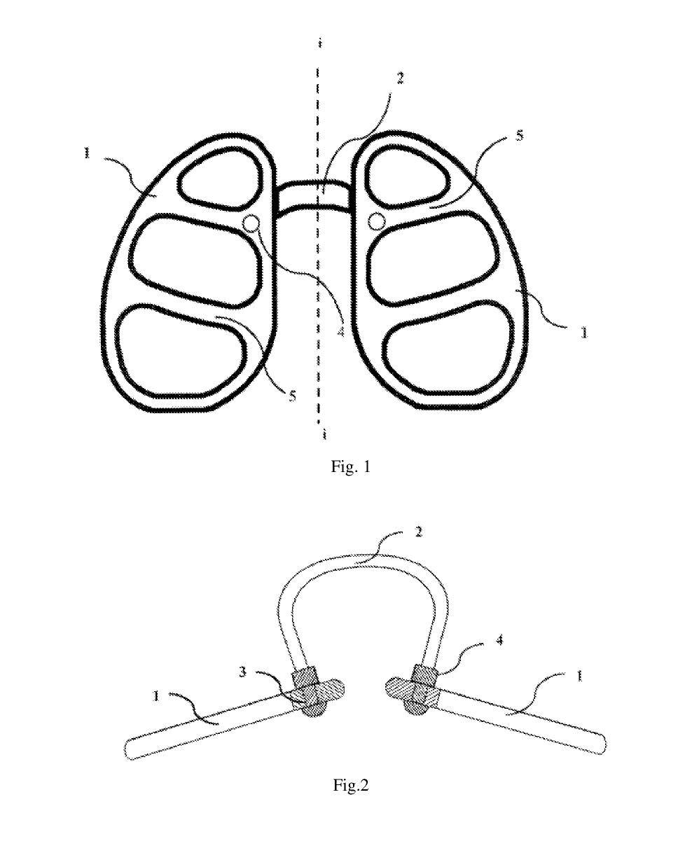

As shown in FIG. 2, the plug-in portion 4 in the embodiment may be a dumbbell-shaped structure which is thin in the middle and thick at both ends. A hole wall of the plug-in hole on the annular stent 1 may be nested around a thinner portion of the plug-in portion 4 in the middle, and thicker portions of the plug-in portion 4 at both ends may be stuck to both sides of the hole wall. The connecting band 2 may be a strip structure which is substantially U-shaped or C-shaped so that an angle between planes on which the two annular stents 1 lie may be 100 degrees to 170 degrees, and the selected angle may progressively increase along with the increase of the cross section of the nasal cavity. After the plug-in portion 4 is inserted into the plug-in hole 3 of the annular stent 1, an edge of a side of the annular stent 1 adjacent to the central axis of the nasal dilator may be spaced apart a distance of 0.5-3 mm from the edge of the plug-in portion 4. The reinforcing rib 5 may be wider at a joint with the inner wall of the annular stent 1 and thinner in the middle.

In addition, in the above embodiments, the nontoxic material may employ plastic, resin, nylon, silicone or compositions fabricated on the basis of these materials.

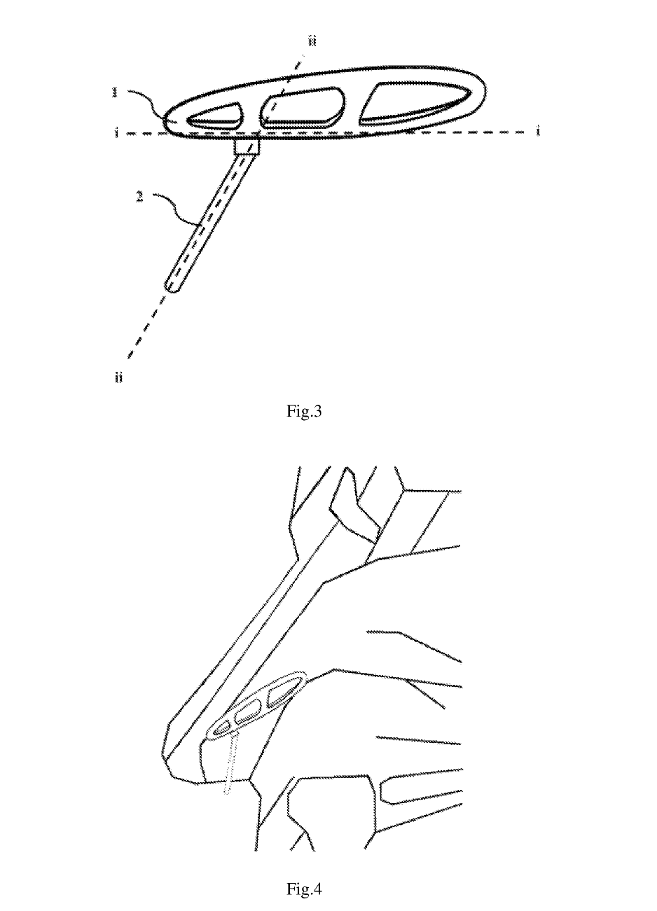

FIG. 4 shows a state in which the nasal dilator shown in FIG. 3 is inserted in a nose. The angle between the central axis (as shown by line ii-ii in FIG. 3) of the connecting band and the central axis of the nasal dilator may progressively increase along with an increase of the cross section of the nasal cavity. To accommodate the shapes of the noses of Asian people, in the present embodiment, the angle between the central axis of the connecting band and the central axis of the nasal dilator may be preferably 55 degrees; the annular stent 1 may be in an inclined posture in the nose so that the connecting band 2 need not to get deeper in the nasal passage, thereby reducing a stimulation to the nasal passage and enabling more comfortable wearing, and a wider side of the annular stent 1 can extend to be adjacent to the nasal cavity small hole deep in the nasal passage.

Finally it is appreciated that the above embodiments are only intended to illustrate technical solutions of the present invention, not intended for limitation purpose. Although the present invention is described in detail with reference to the embodiments, those persons skilled in the art should appreciate that modifications or equivalent substitutes to the technical solutions of the present invention all do not depart from the spirit and scope of the technical solutions of the present invention and all fall within the scope of claims of the present invention.

1. A nasal dilator, comprising: two annular stents and a connecting band, both ends of the connecting band being respectively and symmetrically connected to one annular stent, wherein a plug-in portion is provided at each of both ends of the connecting band, the connecting band is plugged in a plug-in hole on each of the annular stents via the plug-in portions; an central axis of the connecting band is at an angle of 45 degrees to 85 degrees to a central axis of the nasal dilator;
the connecting band is made of a harder nontoxic material and has a height in a range of 8 mm-20 mm;
the annular stent is made of a soft nontoxic material and is a circular ring provided with 1-3 reinforcing rib(s).

2. The nasal dilator according to claim 1, wherein the plug-in portion is in a dumbbell-shaped structure which is thin in the middle and thick at both ends, a hole wall of the plug-in hole on the annular stent is nested around a thinner portion of the plug-in portion in the middle, and thicker portions of the plug-in portion at both ends are stuck to both sides of the hole wall.

3. The nasal dilator according to claim 1, wherein the plug-in hole of the annular stent is located on one side adjacent to the central axis of the nasal dilator.

4. The nasal dilator according to claim 1, wherein after the plug-in portion is inserted into the plug-in hole of the annular stent, an edge of one side of the annular stent adjacent to the central axis of the nasal dilator is spaced apart a distance of 0.5-3 mm from an edge of the plug-in portion.

5. The nasal dilator according to claim 1, wherein the connecting band is in a strip structure which is substantially U-shaped or C-shaped so that an angle between planes on which the two annular stents lie is 100 degrees to 170 degrees.

6. The nasal dilator according to claim 1, wherein a cross section of the annular stent is fit for different shapes of inner cavities of nasal vestibules; a top end of the annular stent is narrower and oriented towards a nose tip when placed in the nasal passage, a bottom end of the annular stent is wider and oriented towards a face when placed in the nasal passage, a side of the annular stent adjacent to the central axis of the nasal dilator is a straight edge or an edge with a small radian and oriented towards the nasal septum when placed in the nasal passage, a side of the annular stent away from the central axis of the nasal dilator is an arcuate edge and oriented towards a nose wing when placed in the nasal passage to support an inner wall of the nasal passage.

7. The nasal dilator according to claim 1, wherein the nontoxic material is selected from a group consisting of plastic, resin, nylon, silicone or compositions fabricated on the basis of these materials.

8. The nasal dilator according to claim 1, wherein the reinforcing ribs are disposed in parallel with the connecting band.

9. The nasal dilator according to claim 1, wherein the reinforcing rib is wider at a joint with an inner wall of the annular stent and thinner in the middle.
